(19) United States
(12) Patent Application Publication
(10) Pub. No.: US 2017/0354417 A1
(43) Pub. Date: Dec. 14, 2017

(54) DEVICE FOR INDUCTION OF CELLULAR ACTIVITY

(71) Applicant: TissueGen, Inc., Dallas, TX (US)

(72) Inventors: Kevin D. Nelson, Richardson, TX (US); Brent B. Crow, Grand Prairie, TX (US); Nickolas B. Griffin, Allen, TX (US); Mario Romero-Ortega, Coppell, TX (US); Jennifer Seifert, Little Elm, TX (US); Nesreen Alzoghoul, Denton, TX (US)

(21) Appl. No.: 15/524,211

(22) PCT Filed: Nov. 16, 2015
(86) PCT No.: PCT/US15/60946

§ 371 Date: May 3, 2017

Related U.S. Application Data
(60) Provisional application No. 62/126,957, filed on Mar. 2, 2015.
(60) Provisional application No. 62/080,302, filed on Nov. 15, 2014.

(51) Int. Cl. A61B 17/11; A61L 27/36; A61K 35/30; A61B 17/00

(52) U.S. Cl. CPC A61B 17/1128; A61L 27/3675; A61K 35/30; A61B 2017/00884; A61B 2017/00893; A61B 2017/1132

(57) ABSTRACT

The present disclosure describes the use of nerve conduits as scaffolds for nerve regeneration, including spinal cord regeneration. The conduit may be hollow or contain a luminal filler such as agar or other biocompatible material.

### CROSS-REFERENCES TO RELATED APPLICATIONS

This application claims the benefit under 35 U.S.C. §119(e) of U.S. Provisional Patent Application No. 62/080,302, filed Nov. 15, 2014, and U.S. Provisional Patent Application No. 62/126,957, filed Mar. 2, 2015, each of which is incorporated herein by reference in their entirety as if fully set forth herein.

### TECHNICAL FIELD OF THE INVENTION

The present disclosure relates generally to the fields of medicine and neurobiology. More particularly, it concerns compositions and methods for the treatment of spinal cord injuries. It also concerns compositions and methods for the generation of small diameter vascular grafts and the healing of complex, multiple tissue injuries such as may occur in battlefield or trauma.

### BACKGROUND OF THE INVENTION

During development and after injury, neural cells migrate and elongate their axons towards proper target cells and organs in response to gradients of biomolecules, which guide axonal regeneration (chemotaxis) either by attachment to the cells or to the extracellular matrix (ECM), or by secretion into the extracellular fluid. In some cases, chemotactic soluble molecules are secreted by specific cells, and gradients are formed through diffusion and convection from the site of release. Cellular responses to such gradients can be influenced by both the nature of the biomolecules such as collagen, fibronectin, and laminin, and physical characteristics of the ECM, such as matrix pore size and stiffness.

In the developing peripheral nervous system (PNS), gradients of neurotrophic factors (NTF) such as nerve-growth factor (NGF), neurotrophin 3 (NT-3), and brain-derived neurotrophic factor (BDNF), are established by distal target cells and direct axonal elongation and target recognition of motor neurons (VMN) from the ventral spinal cord, as well as sensory neurons in the dorsal root ganglia (DRG). In the adult PNS, the efferent branch of sensory neurons re-innervates skin and muscle targets spontaneously after injury, but afferent axons are unable to enter the hostile environment of the adult spinal cord, unless enticed by induced NGF expression.

Pathfinding errors made by injured VMN and DRG neurons during regeneration can be dramatically reduced by establishing the appropriate NTF gradients. Unfortunately, the creation of chemical gradients such as NTF gradients, and the use of chemical gradients in nerve repair remain extremely challenging due to lack of sustained release of desired molecular signals and/or lack of ECM support. Therefore, improved apparatuses and methods for providing a chemical gradient are desired.

### SUMMARY OF THE INVENTION

Thus, in accordance with the present disclosure, there is provided a composition of drug releasing agents arranged in three-dimensional space in such a way to produce one or more concentration gradient(s) of the releasing drug(s). This concentration gradient provides to an organ, tissue, group of cells, or a single cell a means of generating a growth-promoting environment that is capable of initiating and/or sustaining cellular or tissue level responses such as growth, migration, or maturation as directed by the concentration gradient(s) of said drug(s).

The invention applies to a broad range of activities, that can be in vitro, in vivo (in man or other mammal), ex vivo or in silico or other environment or endeavor where guiding or directing cellular or tissue response is desirable.

A method of promoting spinal cord growth or regrowth in a subject is also provided. The method comprises surgically implanting a conduit into a site of spinal cord lesion or deficit of a subject, wherein said conduit is positioned such that the lumen of said conduit is parallel to the axis of desired nerve growth or regrowth. The conduit may be anchored between adjacent vertebral segments, and/or may be held in place by suturing of adjacent neuronal fascia, such as dura mater, or potentially external to the neuronal environment such as muscle fibers. Alternatively, the conduit is not sutured in place. The conduit may be composed of biodegradable polymers, typically alpha-1 esters such as poly-lactide, PLGA, polydioxanone, polyhydroxybuterate, polycaprolactone etc.; other types or classes of polymers may also be utilized such as polyurethane, silicone, cellulose or any member of the modified cellulose family, agarose, collagen, gelatin, or denatured natural extracellular matrix. The spinal cord defect may be congenital, due to trauma, due to infection, due to autoimmune disease, a cervical defect, a lumbosacral defect, a thoracic defect, or it may be surgically induced. The subject may be a human or a non-human mammal. The method may further comprise treating said subject with an anti-inflammatory therapy prior to, at the time of, or post-surgery. The method may further comprise treating said subject with a second therapy prior to or after surgery.

The luminal filler (LF) may be disposed in the lumen of said conduit. The LF may be formed of one or more of agar, collagen, laminin, fibronectin, or any of the glycoprotein family, or any polysaccharide. The LF may comprise microcompartments or microchannels, or may be a single channel, where said single channel may be coincident with and congruent to the entire conduit lumen, containing one or more means to release nerve-growth factors. Whether single or multiple channels or compartments, each such channel or compartment may contain nerve-growth factors comprised in microparticles or eluting fibers disposed in said LF. The nerve-growth factors may elute in a time-release fashion. The surface of the conduit may be coated with one or more nerve-growth factors or biocompatible materials, such as nerve-growth factors that are neurotrophic (NGF, BDNG, NT-3), glial-derived (GDNF) or pleotrophic (PTN, VEGF). The one or more nerve-growth factors may be present in said LF in linear or gradient configurations. The gradient configurations may be unidirectional or bidirectional. The conduit and LF may not comprise any nerve-growth factors.

The conduit may be from about 0.5 mm to about 5 mm in length. In some embodiments, the conduit may have an outer diameter of between about 1.5 mm to about 4.0 mm. In some embodiments, the conduit may have an inner diameter of about 1.5 mm to about 3.0 mm. In some embodiments, the conduit may have a wall thickness of from about 0.2 mm to about 0.6 mm. In some embodiments, the conduit may be rigid. In some embodiments, the conduit may be flexible. In some embodiments, the conduit or implantation site may be treated with collagen.

Also provided is a method of treating a spinal cord deficit in a subject comprising surgically implanting a conduit into a site of spinal cord deficit of a subject, wherein said conduit is positioned such that a lumen of said conduit is parallel to the axons of desired nerve growth or regrowth. Treatment may comprise improving motor control in said subject, or improving sensory function in said subject, such as nociceptive function or mechanoceptive function.

Further provided is a method of treating a nerve or neural tissue deficit in a subject comprising surgically implanting a conduit, medical implant or polymeric regenerative guide fibers into a site of neural deficit of a subject. The neural deficit may be in the brain, spinal cord or peripheral nerve. The brain deficit may be due to, for example, traumatic brain injury or stroke. The medical implant may be an electrode, a deep brain stimulator, a pump, or an antenna.

Embodiments of the disclosed invention are directed to structures/constructs comprising biodegradable fibers that guide and direct cell activity, tissue formation and function, and organ regeneration. In some specialized cases, these constructs can act as cylindrical channels to elicit cell growth/tissue formation within these tubular structures. An embodiment of the invention is directed to axons that can be guided through single or multiple channels using one or more variable pitched coil(s) to provide a standing concentration gradient to these growing axons.

Another embodiment of the invention is directed to using a small diameter vascular graft to demonstrate that specific cells (endothelial cells specifically) can be recruited from surrounding tissue into a device, which will then provide the three-dimensional scaffold from which a functioning tissue is generated. In this application fibers may be placed in weaves at specific locations to create the proper gradients and channels for cell migration and coordination to create a functioning endothelial tissue on the inner lumen of the graft.

In certain embodiments, this concept can be expanded to other tissue types, such as bone and skeletal muscle to show that with the correct choice of growth factors and scaffold construction, cells can be enticed or attracted to specific locations, and can then cooperatively function to form living, vascularized tissue. This application may require single fiber connections to encourage branching. For example, simply creating a tube of skeletal muscle is inadequate, this muscle must be vascularized and innervated to optimally benefit the patient. Therefore, connecting a local functioning vessel to the graft via a VEGF eluting fiber, may induce vascular growth into the skeletal muscles. Similarly, a single fiber or small braid of fibers releasing neurotrophic factors connecting a nearby nerve and then splitting into individual fibers terminating at various places within the skeletal muscle scaffolding may encourage reformation of neuromuscular junctions and could potentially lead to increase function of the graft. This embodiment may pertain to trauma injuries or battlefield injuries where many tissue types are injured and need simultaneous repair.

In the injured adult nervous system, re-establishment of growth- promoting molecular gradients is known to entice and guide nerve repair. However, incorporation of three-dimensional chemotactic gradients in nerve repair scaffolds, particularly in those with single or multi-luminal (ML) architectures, remains extremely challenging. To address this limitation, a method that establishes highly tunable, three-dimensional molecular gradients in nerve guides (NG) by anchoring nerve growth-factor (NGF) releasing coiled microfibers onto the walls of collagen-filled hydrogel microchannels is disclosed. The gradient is achieved by differential pitch in the coiling of neurotrophin-eluting fibers, and in vitro studies demonstrate that axonal growth from dorsal root ganglia (DRG) is 60% longer and more linear as indicated by a reduced turning angle ratio, compared to those exposed to uniform growth factor concentration. Here, we developed a computer model to estimate the dynamics of growth factor release and the diffusion into the luminal collagen, in six different designs of drug release conduits: a) collagen filled NG, b) NGF-Microparticle release in NG, c) NGF-coil release in NG, d) collagen filled ML-NG, e) NGF-Microparticle release in ML-NG, f) NGF-coil release in ML-NG. Finite element computational modeling was used to calculate the spatiotemporal distributions of NGF in the six types of conduits over time, and to compare of growth factor diffusion over time in each of these devices. We further assessed the effect of geometrical parameters on the efficacy of drug release in NGs. Our model provides quantitative insights with time-varying NGF distribution in the microenvironment of the nerve guide conduits. This model will assist in enhancing the design of growth factor secreting nerve conduits, and may improve the current nerve repair strategies to optimize the regeneration of injured neurons and the recovery of function.

An embodiment of the invention is directed to creating a scaffold that can simultaneously repair gapping injuries where various types of tissue, such as bone, blood vessel, nerve all need to be repaired and regenerated. By this point all of the above technologies, small channels with well-maintained gradients, weaves/braids, single fiber work will all be needed to create this type of bridge scaffold.

Embodiments of the inventions are directed to: 1) the ability to create a sustained concentration gradient via a single, or multiple channel lumen with one or more variable-pitched coils coordinated effort of these recruited cells to form functioning tissue; 2) the ability to create a concentration gradient within woven, braided or knitted structures coordinated effort of these recruited cells to form functioning tissue; 3) the ability to induce growth of specific cell types along single fibers including the ability to create branched or bifurcated structures such as nerve and vascular tissue; and 4) coordinated effort of these recruited cells to form functioning tissue.

It is contemplated that any method or composition described herein can be implemented with respect to any other method or composition described herein. The use of the word “a” or “an” when used in conjunction with the term “comprising” in the claims and/or the specification may mean “one,” but it is also consistent with the meaning of “one or more,” “at least one,” and “one or more than one.” It is contemplated that any embodiment discussed in this specification can be implemented with respect to any method or composition of the disclosure, and vice versa. Furthermore, compositions and kits of the disclosure can be used to achieve methods of the disclosure.

Throughout this application, the term “about” is used to indicate that a value includes the inherent variation of error for the device, the method being employed to determine the value, or the variation that exists among the study subjects. The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a device or a method that “comprises,” “has,” “contains,” or “includes” one or more elements possesses those one or more elements, but is not limited to possessing only those one or more elements or steps. Likewise, an element of a device or method that “comprises,” “has,” “contains,” or “includes” one or more features possesses those one or more features, but is not limited to possessing only those one or more features.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A is a schematic representations of a 4 mm complete transection of a spinal cord injury (“SCI”);

FIG. 1B is a schematic representation of an implantation of a Polyurethane Biosynthetic Nerve Implant (“PU-BNI”) into a spinal cord gap;

FIG. 1C is a schematic representation of spinal cord regeneration over the PU tube of FIG. 1B and through agarose microchannels;

FIG. 2A is a photograph of a spinal cord harvested 12 weeks after implantation of a PU-BNI;

FIG. 2B is a photograph of the harvested spinal cord from FIG. 2A with regenerated tissue removed from an outer surface of the PU tube;

FIG. 2C is a photograph of the harvested spinal cord from FIG. 2A with the PU tube removed;

FIG. 3A is a microphotograph of tissue regenerated through a PU-BNI stained for b-tubulin III as a specific marker for neuronal axons;

FIG. 3B is a microphotograph of axonal regrowth over the PU tube of FIG. 3A;

FIG. 3C is a microphotograph of axonal regrowth inside BNI agarose microchannels;

FIG. 4 is a graph showing an average recovery by a BSA treatment group where a 0 on the scale indicates complete paralysis and a 21 is normal locomotive function.

FIGS. 5A and 5B are schematic views of coiled fibers forming molecular gradients in collagen-filled hydrogel microchannels;

FIG. 6 is a schematic view of a TMM casting device showing deployment of fiber coils;

FIGS. 7A, 7B and 7C are schematic views of a Cy3-PLGA fiber coiled onto a metal rod with a gradient formed after deployment in agarose with fluorescence imaging and densitometry;

FIG. 8 is a graph showing BSA release as a loading percentage over time in both high-gradient (H) and low-gradient (L) coiled areas during a 24 hour period;

FIG. 9A is a graph comparing uniform and gradient distribution of coils after one, five, and seven days;

FIG. 9B is a graph of concentration versus microchannel length for uniform and gradient NGF after one, five, and seven days;

FIGS. 10A, 10B and 10C are a collection of multiple confocal images showing deployed polymeric Cy3-coils in agarose gel;

FIG. 11A is a DIC image of a TMM gel showing a microchannel with a DRG explant at one end;

FIG. 11B is a confocal image of DRG axonal growth immunolabeled for β-tubulin visualization within a TMM gel with no coils;

FIG. 11C is a confocal image of DRG axonal growth immunolabeled for β-tubulin visualization within a TMM gel with coils of low-density NGF loaded fibers;

FIG. 11D is a confocal image of DRG axonal growth immunolabeled for β-tubulin visualization within a TMM gel with coils of uniform high-density NGF loaded fibers;

FIG. 11E is a confocal image of DRG axonal growth immunolabeled for β-tubulin visualization within a TMM gel with coils of gradient high-density NGF loaded fibers;

FIG. 11F is a graph showing average axonal length at three NGF concentrations;

FIG. 11G is a graph showing average axonal length for uniform high-density NGF and gradient high-density NGF;

FIG. 12 shows meshed geometries depicting different sizes of mesh elements;

FIGS. 13A, 13B, 13C, 13D, 13E and 13F are a collection of graphs showing axial concentration variation in active channel/microchannel for six designs of a coiled fiber.

### DETAILED DESCRIPTION OF EXEMPLARY EMBODIMENTS

Spinal cord injury (SCI) commonly results in permanent paralysis and sensory impairments due to the lack of spontaneous nerve regeneration in the adult central nervous system. This is even more exacerbated when the injury results in tissue loss as a consequence of trauma. This is normally followed by cell death at the injury epicenter, which results in scar tissue and a fluid filled cyst that prevents axonal regeneration.

A number of strategies have been proposed to repair the injured spinal cord, including soft scaffolds made of degradable polymers such as PLGA, PGE, or multiluminal agarose hydrogels. Most recently such scaffolds will contain growth factors or cells in the lumen to deliver molecules that entice nerve regeneration. Some of these growth factors are neurotropic, such as nerve-growth factor (NGF), brain-derived neurotrophic factor (BDNF) or glial-derived neurotrophic factor (GDNF). The precise method for intraluminal delivery of these growth factors is the focus of intense research.

Here, the inventors have developed a new and surprising therapeutic approach to addressing spinal cord deficits. In a modification of an approach used for treating peripheral nerve deficits, previously considered unsuitable for spinal deficits, the inventors have surprisingly determined that rigid, non-biodegradable conduits can not only be implanted in a site of spinal cord deficit without harm to the subject, but they can promote nerve regrowth both through and over the conduit. Even more surprisingly, this effect appears to be largely independent of the presence or absence of nerve-growth factors. These and other aspects of the disclosure are described in detail below.

### 1. Nerve Defects

A. Spinal Cord Nerve Defects

A spinal cord injury (SCI) or defect is an injury to the spinal cord resulting in a disruption, either temporary or permanent, in the cords normal motor, sensory, or autonomic function. Common causes of damage are trauma (car accident, gunshot, falls, sports injuries, etc.) or disease (transverse myelitis, polio, spinabifida, Friedreich's ataxia, etc.). The spinal cord does not have to be severed in order for a loss of function to occur. Depending on where the spinal cord and nerve roots are damaged, the symptoms can vary widely, from pain to paralysis to incontinence. Spinal cord injuries are described at various levels of “incomplete,” which can vary from having no effect on the patient to a “complete” injury which means a total loss of function.

Treatment of spinal cord injuries starts with stabilizing and immobilizing the spine and controlling inflammation to prevent further damage. The actual treatment can vary widely depending on the location and extent of the injury. In many cases, spinal cord injuries require substantial physical therapy and rehabilitation, especially if the patient's injury interferes with activities of daily life.

Research into treatments for spinal cord injuries includes limiting secondary cellular damage through administration of neuroprotective agents and controlled hypothermia, and inducing nerve regeneration through the use of nerve-growth factors, and stem cells, though many treatments have not been studied thoroughly and very little new research has been implemented in standard care.
i. Classification

The American Spinal Injury Association (ASIA) first published an international classification of spinal cord injury in 1982, called the International Standards for Neurological and Functional Classification of Spinal Cord Injury. Now in its sixth edition, the International Standards for Neurological Classification of Spinal Cord Injury (ISNCSCI) is still widely used to document sensory and motor impairments following SCI. It is based on neurological responses, touch and pinprick sensations tested in each dermatome, and strength of the muscles that control ten key motions on both sides of the body, including hip flexion (L2), shoulder shrug (C4), elbow flexion (C5), wrist extension (C6), and elbow extension (C7). Traumatic spinal cord injury is classified into five categories on the ASIA Impairment Scale:

A indicates a “complete” spinal cord injury where no motor or sensory function is preserved in the sacral segments S4-S5.

B indicates an “incomplete” spinal cord injury where sensory but not motor function is preserved below the neurological level and includes the sacral segments S4-S5. This is typically a transient phase and if the person recovers any motor function below the neurological level, that person essentially becomes a motor incomplete, i.e., ASIA C or D.

C indicates an “incomplete” spinal cord injury where motor function is preserved below the neurological level, and MORE than half of key muscles below the single neurological level of injury have a muscle grade less than 3 (i.e., M0—no contraction, no muscle movement, M1—trace of contraction, but no movement, or M2—movement with gravity eliminated).

D indicates an “incomplete” spinal cord injury where motor function is preserved below the neurological level and at least half of the key muscles (more than 50 percent of the key muscles) below the neurological level have a muscle grade of 3 or more (i.e., M3, M4 or M5, muscle can movement against gravity (3) or with additional resistance (4 & 5)).

E indicates motor and sensation function with ISNCSCI are all graded normal (in all segments) and the patient had neurological deficits from SCI before. Note: only patients with SCI receive any AIS grade. The following incomplete syndromes are not part of the International Standards examination: central cord syndrome, Brown-Sequard syndrome, anterior cord syndrome, cauda equina syndrome, conus medullaris syndrome and all neurological deficits caused by lesion of lower motor neurons, i.e., brachial plexus lesions.
ii. Symptoms

Signs recorded by a clinician and symptoms experienced by a patient will vary depending on where the spine is injured and the extent of the injury. These are all determined by the area of the body that the injured area of the spine innervates. A section of skin innervated through a specific part of the spine is called a dermatome, and spinal injury can cause pain, numbness, or a loss of sensation in the relevant areas. A group of muscles innervated through a specific part of the spine is called a myotome, and injury to the spine can cause problems with voluntary motor control. The muscles may contract uncontrollably, become weak, or be completely paralyzed. The loss of muscle function can have additional effects if the muscle is not used, including atrophy of the muscle and bone degeneration.

A severe injury may also cause problems in parts of the spine below the injured area. In a “complete” spinal injury, all functions below the injured area are lost. An “incomplete” spinal cord injury involves preservation of motor or sensory function below the level of injury in the spinal cord. If the patient has the ability to contract the anal sphincter voluntarily or to feel a pinprick or touch around the anus, the injury is considered to be incomplete. The nerves in this area are connected to the very lowest region of the spine, the sacral region, and retaining sensation and function in these parts of the body indicates that the spinal cord is only partially damaged. This includes a phenomenon known as sacral sparing which involves the preservation of cutaneous sensation in the sacral dermatomes, even though sensation is impaired in the thoracic and lumbar dermatomes below the level of the lesion. Sacral sparing may also include the preservation of motor function (voluntary external anal sphincter contraction) in the lowest sacral segments. Sacral sparing has been attributed to the fact that the sacral spinal pathways are not as likely as the other spinal pathways to become compressed after injury. The sparing of the sacral spinal pathways can be attributed to the lamination of fibers within the spinal cord.

A complete injury frequently means that the patient has little hope of functional recovery. The relative incidence of incomplete injuries compared to complete spinal cord injury has improved over the past half century, due mainly to the emphasis on better initial care and stabilization of spinal cord injury patients. Most patients with incomplete injuries recover at least some function.

Determining the exact “level” of injury is critical in making accurate predictions about the specific parts of the body that may be affected by paralysis and loss of function. The level is assigned according to the location of the injury by the vertebra of the spinal column closest to the injury on the spinal cord:

Cervical. Cervical (neck) injuries usually result in full or partial tetraplegia (Quadriplegia). However, depending on the specific location and severity of trauma, limited function may be retained:
Injuries at the C-1/C-2 levels will often result in loss of breathing, necessitating mechanical ventilators or phrenic nerve pacing.
Injuries at C3 and above typically result in loss of diaphragm function, necessitating the use of a ventilator for breathing.
C4 results in significant loss of function at the biceps and shoulders.
C5 results in potential loss of function at the biceps and shoulders, and complete loss of function at the wrists and hands.
C6 results in limited wrist control, and complete loss of hand function.
C7 and T1 results in lack of dexterity in the hands and fingers, but allows for limited use of arms. Patients with complete injuries above C7 typically cannot handle activities of daily living making functioning independently difficult and not often possible.

Additional signs and symptoms of cervical injuries include inability or reduced ability to regulate heart rate, blood pressure, sweating and hence body temperature. Autonomic dysreflexia or abnormal increases in blood pressure, sweating, and other autonomic responses to pain or sensory disturbances is common.

Thoracic. Complete injuries at or below the thoracic spinal levels result in paraplegia. Functions of the hands, arms, neck, and breathing are usually not affected:
T1 to T8: Results in the inability to control the abdominal muscles. Accordingly, trunk stability is affected. The lower the level of injury, the less severe the effects.
T9 to T12: Results in partial loss of trunk and abdominal muscle control. Typically lesions above the T6 spinal cord level can result in autonomic dysreflexia.

Lumbosacral. The effects of injuries to the lumbar or sacral regions of the spinal cord are decreased control of the legs and hips, urinary system, and anus. Bowel and bladder function is regulated by the sacral region of the spine. In that regard, it is very common to experience dysfunction of the bowel and bladder, including infections of the bladder and anal incontinence, after traumatic injury.

Sexual function is also associated with the sacral spinal segments, and is often affected after injury. During a psychogenic sexual experience, signals from the brain are sent to the sacral parasympathetic cell bodies at spinal levels S2-S4 and in case of men, are then relayed to the penis where they trigger an erection. A spinal cord lesion of descending fibers to levels S2-S4 could, therefore, potentially result in the loss of psychogenic erection. A reflexogenic erection, on the other hand, occurs as a result of direct physical contact to the penis or other erotic areas such as the ears, nipples or neck, and thus not involving descending fibers from the brain. A reflex erection is involuntary and can occur without sexually stimulating thoughts. The nerves that control a man's ability to have a reflex erection are located in the sacral nerves (S2-S4) of the spinal cord and could be affected after a spinal cord injury at this level. The rate of an ejaculation in spinal cord injury varies with the level of the spinal cord injury, with for example complete lesions strictly above Onufs nucleus (S2-S4) being responsive to penile vibratory stimulation in 98% of cases, but in no cases of complete lesion of the S2-S4 segments.

Other syndromes of incomplete injury. Central cord syndrome is a form of incomplete spinal cord injury characterized by impairment in the arms and hands and, to a lesser extent, in the legs. This is also referred to as inverse paraplegia, because the hands and arms are paralyzed while the legs and lower extremities work correctly. Most often the damage is to the cervical or upper thoracic regions of the spinal cord, and characterized by weakness in the arms with relative sparing of the legs with variable sensory loss. This condition is associated with ischemia, hemorrhage, or necrosis involving the central portions of the spinal cord (the large nerve fibers that carry information directly from the cerebral cortex). Corticospinal fibers destined for the legs are spared due to their more external location in the spinal cord.

Ischemia of the spinal cord is reduced blood flow to the spinal cord. Blood flow is supplied by the anterior spinal artery and the paired posterior spinal arteries. This condition may be associated with arterioscleorosis, trauma, emboli, diseases of the aorta, and other disorders. Prolonged ischemia may lead to infarction of the spinal cord tissue. Ischemia of the spinal cord affects its function and can lead to muscle weakness and paralysis. The spinal cord may also suffer circulatory impairment if the segmental medullary arteries, particularly the great anterior segmental medullary artery are narrowed by obstructive arterial disease. When systemic blood pressure drops severely for 3-6 min, blood flow from the segmental medullary arteries to the anterior spinal artery supplying the mid-thoracic region of the spinal cord may be reduced or stopped. These people may also lose sensation and voluntary movement in the areas supplied by the affected level of the spinal cord. This clinical pattern may emerge during recovery from spinal shock due to prolonged swelling around or near the vertebrae, causing pressures on the cord. The symptoms may be transient or permanent.

Anterior cord syndrome is often associated with flexion type injuries to the cervical spine, causing damage to the anterior portion of the spinal cord and/or the blood supply from the anterior spinal artery. Below the level of injury motor function, pain sensation, and temperature sensation are lost, while touch, proprioception (sense of position in space), and sense of vibration remain intact.

Posterior cord syndrome can also occur, but is very rare. Damage to the posterior portion of the spinal cord and/or interruption to the posterior spinal artery causes the loss of proprioception and epicritic sensation (e.g., stereognosis, graphesthesia) below the level of injury. Motor function, sense of pain, and sensitivity to light touch remain intact.

Brown-Sequard syndrome usually occurs when the spinal cord is hemisectioned or injured on the lateral side. True hemisections of the spinal cord are rare, but partial lesions due to penetrating wounds (e.g., gunshot wounds or knife penetrations) are more common. On the ipsilateral side of the injury (same side), there is a loss of motor function, proprioception, vibration, and light touch. Contralaterally (opposite side of injury), there is a loss of pain, temperature, and crude touch sensations. The loss on the contra lateral side begins several dermatome section below the level of injury. This discrepancy occurs because the lateral spinothalamic tracts ascend two or four segments on the same side before crossing.

Tabes Dorsalis results from injury to the posterior part of the spinal cord, usually from infectious diseases such as syphilis, causing loss of touch and proprioceptive sensation. Conus medullaris syndrome results from injury to the tip of the spinal cord, located at the L1 vertebra.
iii. Management

If a suspected spinal cord injury patient is inappropriately or incompletely immobilized, handled, packaged or transported further damage may occur. Deterioration of the initial lesion often occurs during the initial management of injuries; therefore, effective procedures need to be established for the transportation and care to reduce the risk of secondary neurologic damage. A 1988 study estimated that as many as one in four spinal cord injured persons deteriorated between the time of their accident or injury and their arrival in hospital. While some of this is due to the nature of the injury itself, particularly in the case of multiple or massive trauma, some of it reflects the failure to suspect that a spinal injury occurred in the first place and to treat the injured person appropriately.

Health personnel may suspect spinal cord injury in a number of circumstances, in particular if the person: is unconscious as a result of a head injury; has been injured above the clavicle (collarbone) on either side; has been injured in a high-speed motor vehicle accident; or has been injured in any manner known to cause spinal-cord injury.

The first stage in the management of a suspected spinal cord injury follows the basic life support principles of resuscitation. These are represented by the initials DRSABC (which stand for danger, response, send for help, airway, breathing, circulation) but in the context of suspected spinal cord injuries, we add a plus to the A to remind us that we need to look after the airway PLUS add cervical spine control. As a basic principle, the head should be maintained in the neutral position, where spine is not flexed, extended, latterly flexed to either side or rotated. The head should be supported with manual inline support to maintain this position. Traction (pulling on the neck) is not used because injury can be caused by forces which separate the spinal vertebrae and compromise the spinal cord. Critically, the neck is immobilized at, above and below the suspected level of injury, using spinal immobilization equipment. The majority of this management deals with cervical spine injuries, given that they are not only the most common, but being high in the neck they potentially affect all four limbs: in most cases they are therefore the most significant clinically. However, the same principles apply to the thoracic and lumbar spine.

Once the need for resuscitation has been established and attended to if necessary, the person with a suspected spinal cord injury has to be appropriately immobilized. For the first-aider or untrained bystander, this may entail only the positioning of the head in the neutral position and then maintaining it there until more professional help arrives.

This is accomplished with manual inline support (MILS), which is to say holding the head using your hands so that it does not move relative to the body. This may be all that can be done at this stage but represents a significant action in preventing further damage through inadvertent movement of the person prior to a higher level of care being present.

Modern trauma care includes a step called clearing the cervical spine, where a person with a suspected injury is treated as if they have a spinal injury until that injury is ruled out. The objective is to prevent any further spinal cord damage. People are immobilized at the scene of the injury until it is clear that there is no damage to the highest portions of the spine This is traditionally done using a device called a long spine board and a semi-rigid cervical collar, such as an X-Collar, Stifneck, or Wizlock. If the injured person is still inside a vehicle or other confined space, an Extrication Device may be required. This combines a short backboard and flexible, enveloping “wings” which enclose the thorax and are then tensioned using straps, as well as head immobilization device and straps. A minimum of four straps which can be tightened over the person are required to ensure adequate immobilization. A spineboard should not be used without the straps except when sliding an injured person out of an enclosed space or vehicle (in this circumstance it is being used as for rescue or extrication).

Some spineboards are in a single piece, while others that can be scooped under the injured person (or scissored at one end) have locking mechanisms which can be opened and closed to allow the spineboard to be split into two.

The other important piece of equipment used to help mobilize the injured person is the head immobilization device or “headbed.” This device has a base plate which is strapped to the underlying spineboard, and typically two blocks of foam which are placed on either side of the injured person's head. Velcro or adhesive straps are then placed over the top of these blocks to hold the head in position.

If the entire head, neck, and body are appropriately immobilized in this fashion, and the straps tightened to ensure no movement has occurred during the fitting process, it is then appropriate to remove the first responder's hands from providing manual inline support, as the injured person is effectively “packaged” and can be transported knowing that inappropriate movement has been restricted and in most cases eliminated.

A vacuum mattress is a whole-body bean bag mattress that can have the air removed by a pump from within it, leaving a harder outside shell which conforms to the injured person's shape. It is ideally used when an injured person is going to spend a long time during the process of transport as it diminishes the potential for pressure over bony prominences while lying face up.

There are arguments in medical literature about the efficacy of collars, spineboards, and head immobilization devices. It is important to ensure they are properly applied as they can then provide a more secure method of transporting an injured person. The alternative is requiring a first responder to stay at the head of the injured person and apply manual in line support for what may be a great deal of time and maintain vigilance when moving the injured person, loading them into and out of an ambulance and accompanying all the way into a hospital.

Before the protective cervical collar is removed, the spine must be “cleared,” which is to say the potential for instability and (further) damage to the delicate spinal cord eliminated. This is usually done according to a protocol derived from studies of spinal injury, including the NEXUS and Canadian C Spine studies. Techniques of immobilizing the affected areas in the hospital include Gardner-Wells tongs, which can also exert spinal traction to reduce a fracture or dislocation.

One experimental treatment, therapeutic hypothermia, is used but there is no evidence that it improves outcomes. Maintaining mean arterial blood pressures of at least 85 to 90 mmHg using intravenous fluids, transfusion, and vasopressors to ensure adequate blood supply to the spinal cord and preventing damage to the spinal cord is another treatment with little evidence of effectiveness. Surgery may also be necessary to remove any bone fragments from the spinal canal and to stabilize the spine.

Inflammation can cause further damage to the spinal cord, and patients are sometimes treated with drugs to reduce swelling. Corticosteroid drugs are used within 8 hours of the injury. This practice is based on the National Acute Spinal Cord Injury Studies (NASCIS) I and II, though other studies have shown little benefit and concerns about side effects from the drug have changed this practice. High dose methylprednisolone may improve outcomes if given within 6 hours of injury. However, the improvement shown by large trials has been small, and comes at a cost of increased risk of serious infection or sepsis due to the immunosuppressive qualities of high-dose corticosteroids. Methylprednisolone is no longer recommended in the treatment of acute spinal cord injury.

Patients often require extended treatment in specialized Spinal Unit or an intensive care unit. When treating a patient with a SCI, repairing the damage created by injury is the ultimate goal. By using a variety of treatments, greater improvements are achieved, and, therefore, treatment should not be limited to one method. Furthermore, increasing activity will increase chances of recovery. The rehabilitation process following a spinal cord injury typically begins in the acute care setting. Physical therapists, occupational therapists, nurses, social workers, psychologists and other health care professionals typically work as a team under the coordination of a physiatrist to decide on goals with the patient and develop a plan of discharge that is appropriate for the patient's condition.

In the acute phase, physical therapists focus on the patient's respiratory status, prevention of indirect complications (such as pressure ulcers), maintaining range of motion, and keeping available musculature active. Also, there is great emphasis on airway clearance during this stage of recovery. Following a spinal cord injury, the individual's respiratory muscles may become weak, making the patient unable to cough effectively and allowing secretions to accumulate within the lungs. Physical therapy treatment for airway clearance may include manual percussions and vibrations, postural drainage, respiratory muscle training, and assisted cough techniques. Patients are taught to increase their intra-abdominal pressure by leaning forward to induce cough and clear mild secretions. The quad cough technique is done with the patient lying on their back and the therapist applies pressure on their abdomen in the rhythm of the cough to maximize expiratory flow and mobilize secretions. Manual abdominal compression is another effective technique used to increase expiratory flow which later improves cough. Other techniques used to manage respiratory dysfunction include respiratory muscle pacing, use of an abdominal binder, ventilator-assisted speech, and mechanical ventilation.

Depending on the neurological level of impairment (NLI), the muscles responsible for expanding the thorax, which facilitate inhalation, may be affected. If the NLI is such that it affects some of the ventilatory muscles, more emphasis will then be placed on the muscles with intact function. For example, the intercostal muscles receive their innervation from T1-T11, and if any are damaged, more emphasis will need to placed on the unaffected muscles which are innervated from higher levels of the CNS. As SCI patients suffer from reduced total lung capacity and tidal volume physical therapists teach SCI patients accessory breathing techniques (e.g., apical breathing, glossopharyngeal breathing, etc.) that typically are not taught to healthy individuals.

B. Brain Nerve Deficits

Cranial nerve disease is an impaired functioning of one of the twelve cranial nerves. It is possible for a disorder of more than one cranial nerve to occur at the same time, if a trauma occurs at a location where many cranial nerves run together, such as the jugular fossa. A brainstem lesion could also cause impaired functioning of multiple cranial nerves, but this condition would likely also be accompanied by distal motor impairment.

The facial nerve is the seventh of 12 cranial nerves. This cranial nerve controls the muscles in the face. Facial nerve palsy is more abundant in older adults than in children and is said to affect 15-40 out of 100,000 people per year. This disease comes in many forms which include congenital, infectious, traumatic, neoplastic, or idiopathic. The most common cause of this cranial nerve damage is Bell's palsy (idiopathic facial palsy) which is a paralysis of the facial nerve. Although Bell's palsy is more prominent in adults it seems to be found in those younger than 20 or older than 60 years of age. Bell's Palsy is thought to occur by an infection of the herpes virus which may cause demyelination and has been found in patients with facial nerve palsy. Symptoms include flattening of the forehead, sagging of the eyebrow, and difficulty closing the eye and the mouth on the side of the face that is affected. The inability to close the mouth causes problems in feeding and speech. It also causes lack of taste, lacrimation, and sialorrhea.

Brain damage or brain injury (BI) is the destruction or degeneration of brain cells, including nerves. Brain injuries occur due to a wide range of internal and external factors. A common category with the greatest number of injuries is traumatic brain injury (TBI) following physical trauma or head injury from an outside source, and the term acquired brain injury (ABI) is used in appropriate circles to differentiate brain injuries occurring after birth from injury due to a disorder or congenital malady.

In general, brain damage refers to significant, undiscriminating trauma-induced damage, while neurotoxicity typically refers to selective, chemically induced neuron damage. Brain injuries occur due to a very wide range of conditions, illnesses, injuries, and as a result of iatrogenesis (adverse effects of medical treatment). Possible causes of widespread brain damage include birth hypoxia, prolonged hypoxia (shortage of oxygen), poisoning by teratogens (including alcohol), infection, and neurological illness. Chemotherapy can cause brain damage to the neural stem cells and oligodendrocyte cells that produce myelin. Common causes of focal or localized brain damage are physical trauma (traumatic brain injury, stroke, aneurysm, surgery, other neurological disorder), and poisoning from heavy metals including mercury and its compounds of lead.

C. Peripheral Nerve Deficits

Peripheral nerve damage is categorized in the Seddon classification based on the extent of damage to both the nerve and the surrounding connective tissue since the nervous system is characterized by the dependence of neurons on their supporting glia. Unlike in the central nervous system, regeneration in the peripheral nervous system is possible. The processes that occur in peripheral regeneration can be divided into the following major events: Wallerian degeneration, axon regeneration/growth, and nerve reinnervation. The events that occur in peripheral regeneration occur with respect to the axis of the nerve injury. The proximal stump refers to the end of the injured neuron that is still attached to the neuron cell body; it is the part that regenerates. The distal stump refers to the end of the injured neuron that is still attached to the end of the axon; it is the part that will degenerate but remains the area that the regenerating axon grows toward.

The lowest degree of nerve injury in which the nerve remains intact but its signaling ability is damaged is called neurapraxia. The second degree in which the axon is damaged, but the surrounding connecting tissue remains intact is called axonotmesis. The last degree in which both the axon and connective tissue are damaged is called neurotmesis.

### 2. Nerve Growth Scaffolds

In accordance with the present disclosure, the inventors contemplate inserting supports into the site of spinal cord deficits in order to provide a scaffold upon and through which spinal cord axons may regrow. The scaffolds may be combined with other features, such as various biological factors that may stimulate, promote or improve nerve growth.

A. Conduits

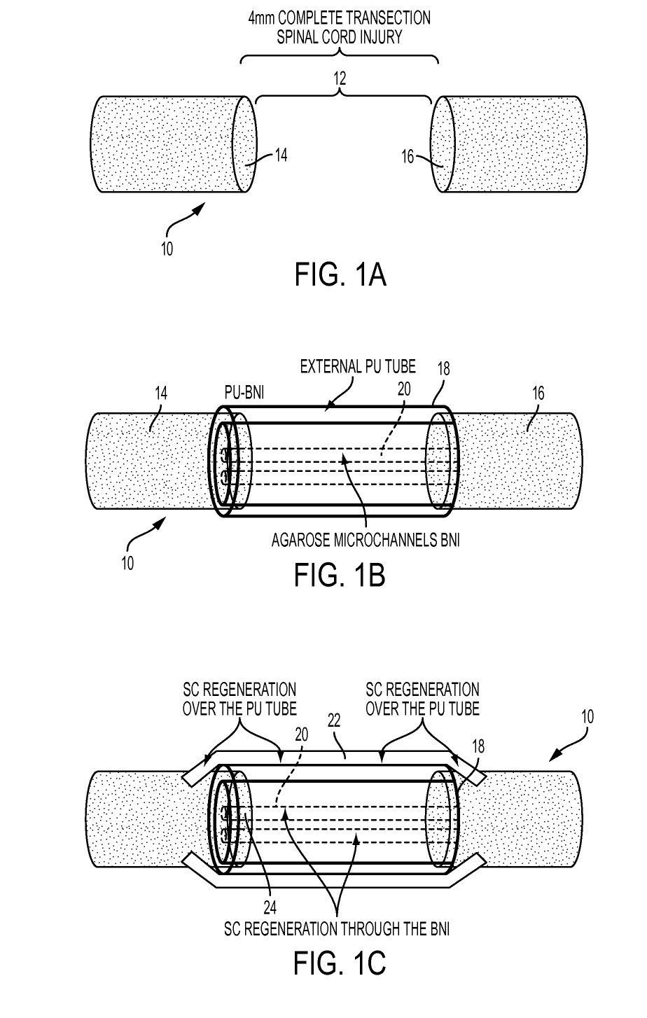

The scaffolds of the present disclosure are exemplified by conduits that are defined by elongated tubular structures with open ends and a lumen passing therethrough. FIGS. 1A-1C are schematic representations of an implantation of a tube into a spinal cord gap. FIG. 1A shows a spinal cord 10 with a gap 12 disposed between a first spinal cord portion 14 and a second spinal cord portion 16. FIG. 1B shows a tube 18 placed in the gap 12. In some embodiments, the tube 18 may be a Polyurethane-Biosynthetic Nerve Implant (“PU-BNI”). The tube 18 is a tube comprising a circular cross-section and one or more microchannels 20 that extend along a length of the tube 18 in an axial direction. FIG. 1C demonstrates a regenerated tissue 22 that has formed around an exterior of the tube 18 and a regenerated tissue 24 that has formed through the microchannels the one or more microchannels 20.

While the exemplified tube 18 has a circular cross-section, other cross-sectional shapes are also suitable, such as oval, square, rectangular or hexagonal. The tube 18 may be made from materials that are rigid to semi-rigid in nature, sustaining a force of 100 kPa to 2.0 GPa. The tube 18 may be non-biodegradable, or at least not biodegradable for months to years following implantation.

In some embodiments, the tube 18 may be formed from, for example, poly-lactide acid (“PLA”), poly-urethane, silicone, cellulose, collagen, poly-lactide co-glycolic acid, polycaprolactone, or denatured natural extracellular matrix. In some embodiments, the tube 18 may have a length of about 0.5 mm to about 5 mm, an outer diameter of about 1.5 mm to about 4.0 mm, and a lumen diameter of about 1.5 mm to about 3.0 mm. In some embodiments, the tube 18 wall may have a thickness of about 0.2 mm to about 0.6 mm.

In some embodiments, tube 18 can be formed using 0.037″×0.027″ Micro-Renathane®, a commercially available blood-compatible tubing. In some embodiments, the polyurethane tubing does not contain any plasticizers, antioxidants, tints or colorants, and is hydrolytically stable and is unaffected by most non-polar solvents and medical solutions.

In some embodiments, the tube 18 may further be coated with a biocompatible material or nerve-growth factors, as discussed below. For example, the tube 18 may be coated with collagen and other extracellular matrix components.

B. Polymer Fibers

In another embodiment, the scaffold will be composed of polymer fibers that act as a regenerative guide for growing/regrowing nerve tissue. The fibers will act as a more traditional scaffold, with nerves growing on top or around the scaffold. Suitable polymers include poly-lactide acid, poly-urethane, silicone, cellulose, collagen, poly-lactide co-glycolic acid, and polycaprolactone.

C. Biosynthetic Nerve Implants

In some embodiments, a conduit may contain an element variably termed a biosynthetic nerve implant or a luminal filler. This element may be a solid, semi-solid, or gel that can provide further support for the growth of nerve tissue, as well as a depot for the delivery of growth factors (discussed below). Suitable materials for the luminal filler include agar, collagen, laminin, fibronectin, or glycoproteins.

In some embodiments, the filler can be of uniform nature or can be made to contain a differential concentration of molecules such as collagen, laminin, fibronectin, growth factors, biopolymers, and pharmacological agents such as the anti-inflammatory molecule dexamethasone. In some embodiments, the filler may be solid, or may contain microparticles, microcompartments or microchannels, again to facilitate growth of new nerve tissue through the conduit, and to act as a repository for factors.

In some embodiments, microcompartments in the lumen can be in turn filled with collagen, polymeric microparticles, or fibers and/or cells, such as Schwann cells, fibroblasts, stem cells, induced pluripotent cells (IPCs), or other supportive cells. These cells can be either from autologous sources or genetically modified to express molecules that can facilitate axonal regeneration such as growth factors.

In some embodiments, microcompartments can be used to provide a controlled environment for the cells cultured in it prior to implantation or those migrating into it after implantation. This environment can consist in incorporating diverse means for the sustained delivery of growth factors, cytokines, and anti-inflammatory molecules.

Among the molecules incorporated into the tube or the Biosynthetic Nerve Implant (“BNI”) can be blockers for growth inhibitory molecules including those designed to block myelin-associated inhibitors (MAG and EphB3), and the chondroitin sulphate proteoglycans (CSPG) versican and neurocan.

D. Nerve-Growth Factors

In some embodiments, the biosynthetic nerve implant may be designed to deliver a growth factor. Alternatively, the conduit itself may be coated with a nerve-growth factor. These factors may be neurotrophic (NGF, BDNF, NT-3), glial-derived (GDNF) or pleotrophic (PTN, VEGF).

Nerve-growth factor (NGF) is a small secreted protein that is important for the growth, maintenance, and survival of certain target neurons (nerve cells). It also functions as a signaling molecule.

While “nerve-growth factor” refers to a single factor, “nerve-growth factors” refer to a family of factors also known as neurotrophins. Members of the neurotrophin family well recognized for their growth promoting effect include: Nerve-growth factor (NGF), Brain-Derived Neurotrophic Factor (BDNF), Neurotrophin-3 (NT-3), and Neurotrophin 4/5 (NT-4/5). BDNF is a protein that is encoded by the BDNF gene. BDNF binds at least two receptors on the surface of cells that are capable of responding to this growth factor, TrkB and the LNGFR (low-affinity nerve-growth factor receptor, also known as p75). It may also modulate the activity of various neurotransmitter receptors, including the Alpha-7 nicotinic receptor. BDNF has also been shown to interact with the reelin signaling chain. Neurotrophin-3 is a protein that is encoded by the NTF3 gene. It has activity on certain neurons of the peripheral and central nervous system, which helps to support the survival and differentiation of existing neurons, and encourages the growth and differentiation of new neurons and synapses. Neurotrophin-4 (NT-4), also known as neurotrophin-5 (NT-5) or NT-4/5, is encoded by the NTF4 gene. NT-4 is a neurotrophic factor that signals predominantly through the TrkB receptor tyrosine kinase.

The GDNF family of ligands (GFL) consists of four neurotrophic factors: glial cell line-derived neurotrophic factor (GDNF), neurturin (NRTN), artemin (ARTN), and persephin (PSPN). GFLs have been shown to play a role in a number of biological processes including cell survival, neurite outgrowth, cell differentiation and cell migration. In particular signaling by GDNF promotes the survival of dopaminergic neurons and potently promotes the survival of many types of neurons.

Pleiotrophin (PTN) also known as heparin-binding brain mitogen (HBBM) or heparin-binding growth factor 8 (HBGF-8), neurite growth-promoting factor 1 (NEGF1), heparin affinity regulatory peptide (HARP), or heparin binding growth associated molecule (HB-GAM), is encoded by the PTN gene. It is an 18-kDa growth factor that has a high affinity for heparin. It is structurally related to midkine and retinoic acid induced heparin-binding protein.

Vascular endothelial growth factor (VEGF), originally known as vascular permeability factor (VPF), is a signal protein produced by cells that stimulates vasculogenesis and angiogenesis. It is part of the system that restores the oxygen supply to tissues when blood circulation is inadequate. Serum concentration of VEGF is high in bronchial asthma and diabetes mellitus. VEGF's normal function is to create new blood vessels during embryonic development, new blood vessels after injury, muscle following exercise, and new vessels (collateral circulation) to bypass blocked vessels.

PCT/US14/16905 and US20070010831 also describe growth factors for use in nerve regeneration. The contents of these applications are hereby incorporated by reference.

### 3. Surgical Procedures

Suitable recipients of the procedure include humans and non-human animals, such as chimps, monkeys, dogs, cats, horses, pigs, cows, goats, sheep, mice, rats, and rabbits.

Patients with an injured human spinal cord will be evaluated by a number of different criteria in order to determine whether they can benefit from the present procedure including time after injury, affected level and whether they present a complete, incomplete or discomplete lesion.

Since any experimental intervention presents risks, patients with acute lesions in the thoracic region will be likely candidates to test this technology, where only marginal functional consequences are at risk, and the benefit for restoring motor and bowel functions are maximized. After obtaining safety and efficacy data from clinical trials of treatment in thoracic lesions, one can then move to higher lesions and into more chronic injuries.

The best treatment option will likely comprise conduits with BNI luminal fillers containing well-defined molecular attractants that are axon type specific depending on the dorso-ventral level of the injury. They will also contain a programmable and sustained release of growth inhibitor blockers. The recovery of function can be also stimulated by physical rehabilitation therapy, neuromodulation therapy or a combination thereof.

### 4. EXAMPLES

The following examples are included to demonstrate particular embodiments of the disclosure. It should be appreciated by those of skill in the art that the techniques disclosed in the examples which follow represent techniques discovered by the inventor to function well in the practice of the disclosure, and thus can be considered to constitute specifically contemplated modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

A 2-3 mm injury gap was introduced into the spinal cord tissue of 53 adult female Long Evans rats by severing and aspiration after a laminectomy at T9-11. The bottom of the vertebra was scraped with a blade to eliminate any possible connections, resulting in a drastic gap injury that would likely never recover spontaneously.

Next, a biosynthetic nerve implant (“BNI”) was introduced. The BNI was surrounded by a rigid Polyurethane (“PU”) tube (e.g., the tube 18 from FIGS. 1B and 1C). The PU tube, which was relatively thick, suturable, and non-biodegradable, was filled with a 1.5% agarose plug. Microchannels (e.g., the one or more microchannels 22 from FIGS. 1B and 1C) were cast in the BNI using linearly placed metal rods in the agarose. This approach, which has heretofore only been used in peripheral nerve injury models, is described in U.S. Patent Publication No. 20070010831. Unlike a peripheral nerve, a spinal cord cannot be sutured as it is composed by very soft tissue, and previous protocols for these model injuries removed the PU tube, with only the multiluminal agarose being implanted. However, in this case, the agarose plug with the PU tube in place was anchored into the spinal column by inserting ends of the PU tube under the immediately adjacent proximal and distal vertebral bodies. The implanted PU tube was then covered with collagen type I gel and the muscles were sutured back in place.

### Example 2—Results

52 rats received some version of a PU-BNI, with the following assignments of animals to test groups:

8 sham (no implant);
8 with BSA gradients by luminal variable pitch coils;
8 with gradients of BSA (ventral) and BDNF (dorsal) by luminal, variable-pitched coils;
8 with gradients of GDNF (ventral) and BSA (dorsal) by luminal, variable-pitched coils;
12 with gradients of GDNF (ventral) and BDNF (dorsal) by luminal, variable-pitched coils (4 animals were originally planned for a 4-week survival, but were later merged with the rest of the group); and
8 with gradients of BDNF (ventral) and GDNF (dorsal) by luminal, variable-pitched coils.

The study was initially planned for 9 weeks, but was extended to 12 weeks after encouraging behavioral data prior to the 9 week period. After taking into account the rats lost during the study to morbidity and mortality, the inventors were left with the following number of animals in each group from which usable data (at least 8 weeks of behavior data) was obtained:

7 sham (no implant);
6 with BSA gradients by luminal variable pitch coils;
5 with gradients of BSA (ventral) and BDNF (dorsal) by luminal, variable-pitched coils;
7 with gradients of GDNF (ventral) and BSA (dorsal) by luminal, variable-pitched coils;
7 with gradients of GDNF (ventral) and BDNF (dorsal) by luminal, variable-pitched coils (4 animals were originally planned for a 4-week survival, but were later merged with the rest of the group);
6 with gradients of BDNF (ventral) and GDNF (dorsal) by luminal, variable-pitched coils; and
5 variable-pitched coils.

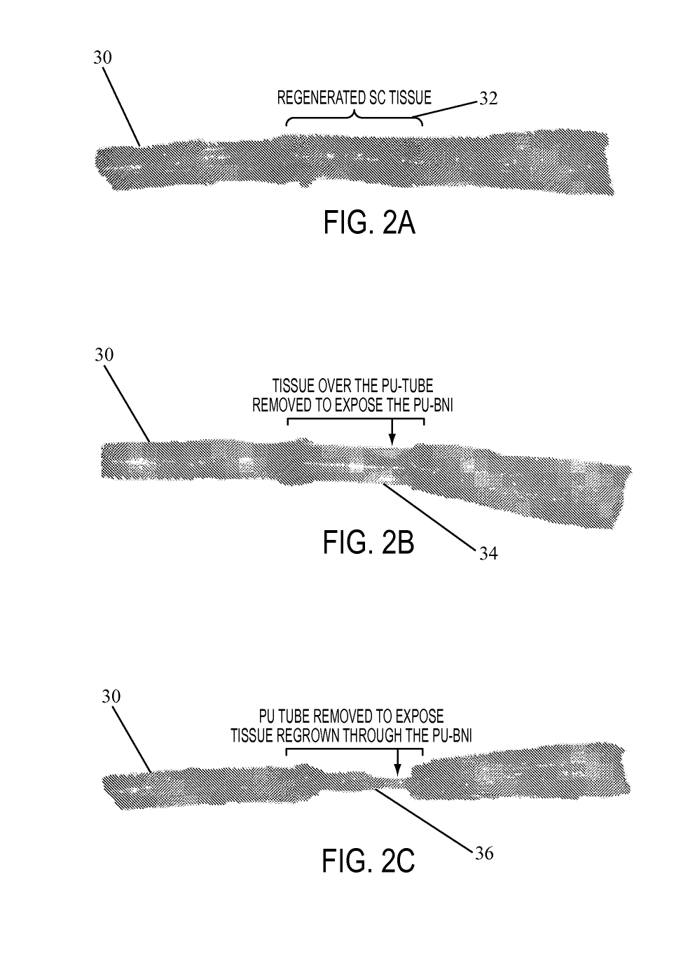

At the end of the study, animals were examined and the spinal cord tissue evaluated at the injury site. Surprisingly, the spinal tissue regenerated in the initial 5 animals examined to the point that in some, localizing the injury site was very difficult. In some animals, the tube dislodged and was found over the spinal cord; however the cord still regenerated, and tissue was seen connecting the cord to the agarose channels in the tube. Exemplary results are shown in FIGS. 2A-2C. FIG. 2A is a photograph of a harvested spinal cord 30 that was removed from a subject after 12 weeks. FIG. 2A shows regenerated spinal cord 32 has grown over an implanted PU tube 34 (best seen in FIG. 2B), covering the PU tube 34 completely. Incredibly, the gross anatomy of this tissue looked normal. FIG. 2B is a photograph of the spinal cord 30 with the regenerated spinal cord 32 removed to expose the PU tube 34. FIG. 2C is a photograph of the spinal cord 30 with the regenerated spinal cord 32 and the PU tube 34 removed to show regenerated spinal cord 36 that has grown through the PU tube 34.

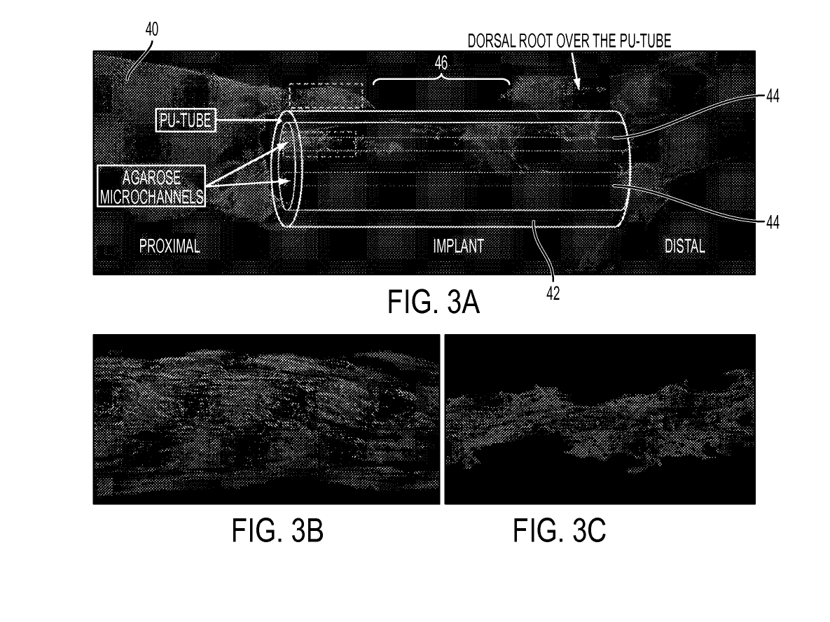

Histological evaluation of the regenerated tissue using a specific antibody for axons (b-tubulin III) revealed that neural tissue grew over the PU tube to the point that one could identify what appear to be dorsal roots emanating from the cord tissue growing on top of the PU tube as well as through the lumen of the BNI microchannels. For example, see FIG. 3A. FIG. 3A is a microphotograph of a spinal cord 40 removed from an adult rat. An outline of a PU tube 42 has been superimposed onto FIG. 3A to better show the positioning of the PU tube 42 relative to the spinal cord 40. The PU tube 42 includes an outer surface and a lumen. Two microchannels 44 are shown disposed within the lumen of the PU tube 42. Dorsal roots 46 are shown emanating from the cord tissue and growing over the outer surface of the PU tube 42. FIG. 3B is a microphotograph showing axon regrowth over the PU tube 42. FIG. 3C is a microphotograph showing axon regrowth inside the PU tube 42. This result confirms the permissive nature of the PU tube 42 and of BNI multiluminal fillers, which may be placed within the microchannels 44, in repairing a complete transection of the spinal cord 40.

In order to confirm that the observed neural growth have not only regenerated across the injury site, but reached the more distal lumbar segments that control sensori-motor function of the hindlimb, the inventors performed an open field behavioral test of locomotor activity and assigned score based on a widely accepted scale known as the Basso, Beattie, and Bresnahan (BBB) scale. The BBB scale provides evidence of the sequence of locomotor recovery patterns and takes into consideration the early (BBB score from 0 to 7), intermediate (8-13) and late phases (14-21) of recovery (Basso et al., 1995).

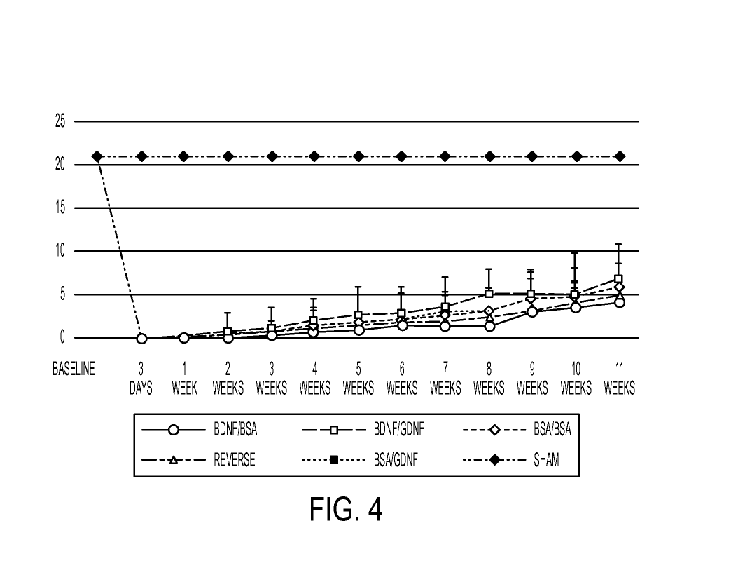

In our study, all injured animals showed complete paralysis for 2 weeks following the lesion to the spinal cord. Thereafter, all groups containing either the neutral bovine serum albumin (BSA) control, as well as those containing sustained release of several growth factors including BDNF and GDNF combined with themselves or with BSA, showed progressive improvement of hindlimb function. FIG. 4 is a graph showing average recovery by a BSA treatment group where a 0 on the scale indicates complete paralysis and a 21 is normal locomotive function. At the end of 11 weeks after the PU-BNI implantation, the BSA group showed an average BBB sore of 6 which is a strong indicator of early locomotor recovery.

### Example 3

### Spinal Cord Injury and Implantation

52 adult female Long Evans rats were implanted with the spinal cord biosynthetic nerve implant (BNI) and divided into the following seven experimental groups: a) Sham injury (n=8), b) gradients of BSA by luminal, variable pitch coils (n=8), c) gradients of BSA (ventral) and BDNF (dorsal) by luminal, variable-pitched coils (n=8), d) gradients of BSA (dorsal) and GDNF (ventral) by luminal, variable-pitched coils (n=8), e) gradients of GDNF (ventral) and BDNF (dorsal) by luminal, variable-pitched coils but where the coil is placed with the gradient inverted, i.e. BDNF gradient increases moving towards the tail and GDNF coil will also be put in backwards, i.e. gradient increase towards the head (n=8), and f) gradients of GDNF (ventral) and BDNF (dorsal) by luminal, variable-pitched coils for a 4-week survival study (n=4)) and an additional n=8 of the same BNI configuration but part of the 9 week study. Gross anatomical evaluation of nerve regeneration was performed at 12 weeks post implantation.

### Electrophysiology

In some animals, recording of transcranial motor-evoked potentials (TcMEPs) was obtained following anesthesia induction and just prior (within 5 minutes) to the first incision (baseline), and after the transection of the spinal cord. Terminal electrophysiology was also obtained at the end of the study to determine how electro-competent the spinal cord is following recovery.

### Behavioral Testing

Behavioral testing: Locomotion of each animal was evaluated for motor functional recovery by the BBB rating scale for gait analysis.

### Tract Tracing

Prior to histochemical analysis, the sensory and the corticospinal axons were labeled with 1% Cholera Toxin B (CTB) and 10% Biotin Dextran Amine (BDA), respectively. This allowed the extent of regeneration and reinnervation to be evaluated.

### Histology

Spinal cord sections from each group were stained to evaluate axonal content in the dorsal and ventral regions. The amount of nerve regeneration was quantified and statistically evaluated.

### RESULTS

Surgical Description of Implantation of the Biosynthetic Nerve Implant (BNI) into the Spinal Cord

Female Long-Evans rats (225-275 g) were used for this study. The animals were anesthetized with sodium pentobarbital (50 mg/kg; intraperitoneal). When an adequate depth of anesthesia was attained (loss of corneal reflex), the shaved dorsal surface was cleaned with povidone-iodine. The vertebral column was exposed by dissection of the paraspinous muscles and laminectomies performed at T10 and T11. The spinal cord was completely transected in two places, approximately 2 mm apart, to create a gap injury. Once transected, the spinal cord stumps retracted, resulting in a gap of approximately 4 mm. The BNI was placed into the gap, approximating the remaining ends of the spinal cord. A liquid solution of collagen was added on top of the implant and allowed to polymerize in place. A section of subcutaneous adipose tissue was then placed over the collagen and the incision closed. 4-0 chromic gut was utilized to close the muscle layers and the skin was approximated and closed with wound clips.

### CONCLUSIONS

The biological activity of the gradient was confirmed in dorsal root ganglia explant cultures. Axonal length increased significantly when exposed to a gradient of growth factor as compared to non-gradient coils, despite the same number of turns. Gradient release of NGF enhanced the directionality of the axonal outgrowth. Different configurations produce different spatiotemporal concentrations for same reference 100 ng/ml initial reference concentration. Axon straightness protocol can be used to predict the linearity of axons in different designs. For coiling configuration, axon linearity increases with differential coiling and decreasing channel size.

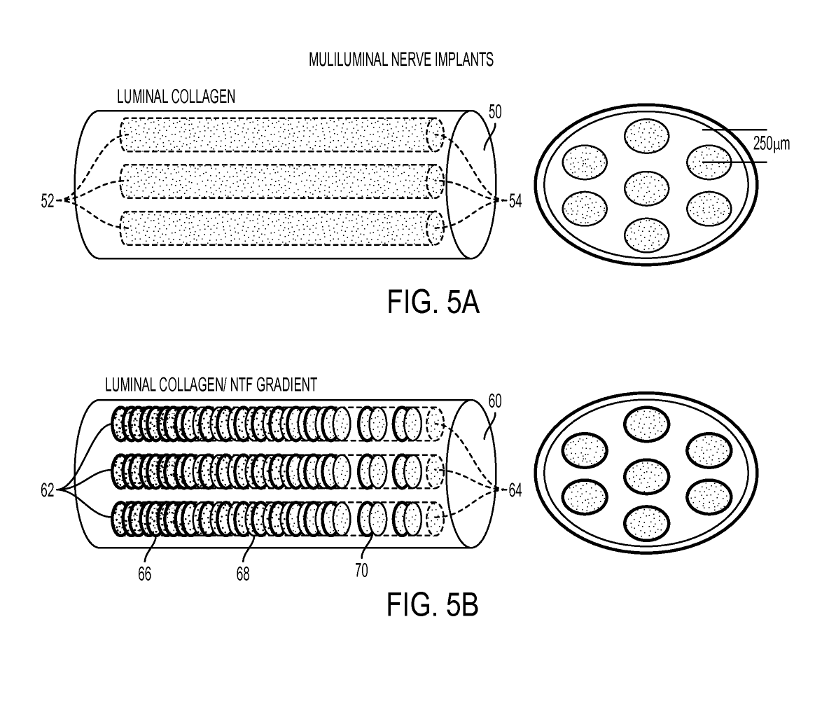

FIGS. 5A and 5B illustrate a tube having luminal collagen having neurotrophic factor (NTF) gradients compared to a tube with luminal collagen having NTF without gradients. A first tube 50 is shown comprising multiple luminal collagen 52 disposed within multiple microchannels 54. A second tube 60 is shown comprising multiple luminal collagen 62 disposed in multiple microchannels 64. In contrast to the luminal collagen 52, the luminal collagen 62 comprises NTF arranged to comprise a density gradient along a length of the collagen 62. For example, a portion 66 comprises a relatively higher density than a portion 68, and the portion 68 comprises a relatively higher density than a portion 70.

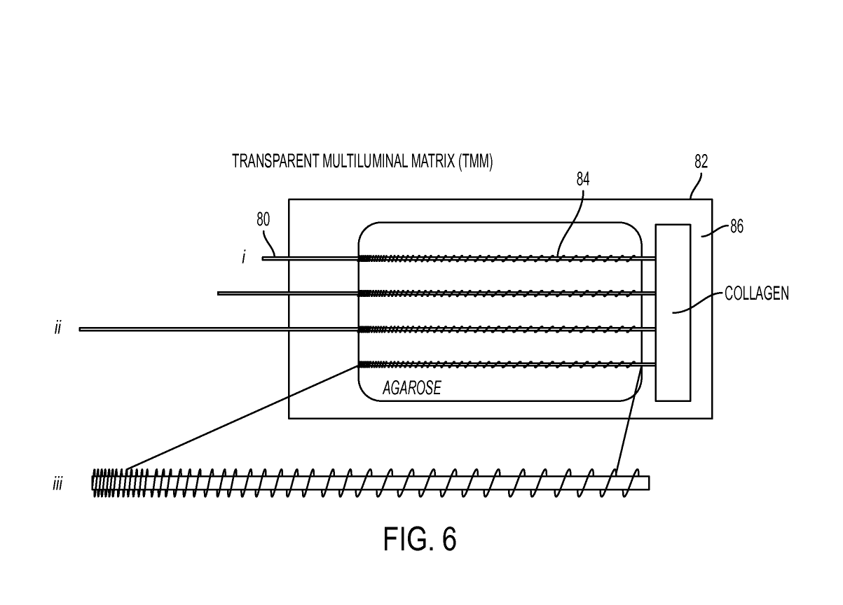

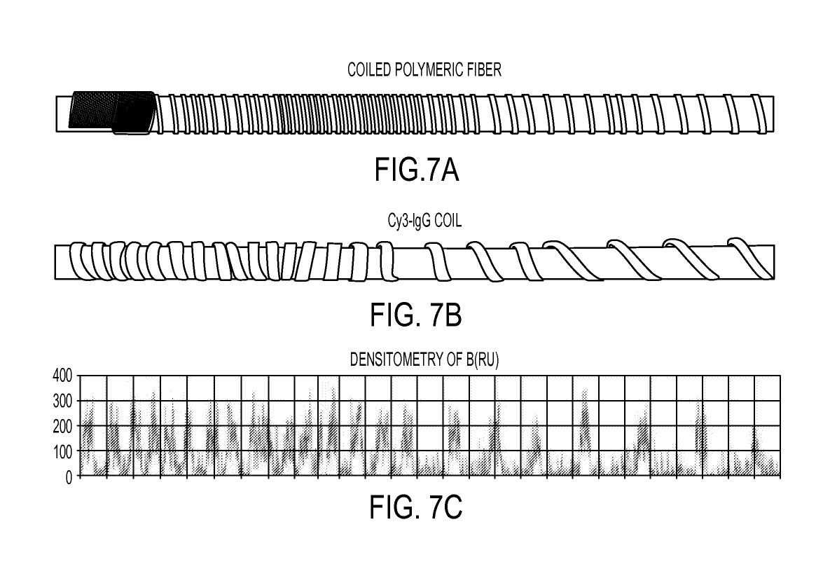

FIG. 6 shows stages of a process for deploying fiber coils in a microchannel with a casting device. At stage (i), a metal rod 80 is shown inserted in a casting device 82. The casting device 82 includes a casting channel 84. As shown in stage (i), agarose polymerization has already occurred. At stage (ii), the metal rod 80 has been partially withdrawn from the casting channel 84, which draws collagen from a collagen reservoir 86 into the casting channel 84. Stage (iii) shows the metal rod 80 removed from the casting channel 84. Removal of the metal rod 80 from the casting device 82 after agarose polymerization anchors the coils onto the walls of the microchannel while simultaneously filling the lumen with collagen. FIG. 7 shows: A) a Cy3-PLGA fiber coiled onto a metal rod (e.g., such as the metal rod 80 from FIG. 6); B) a fluorescence image of a Cy3-PLGA fiber coiled onto a metal rod; and C) a densitometry graph of B (Ru).

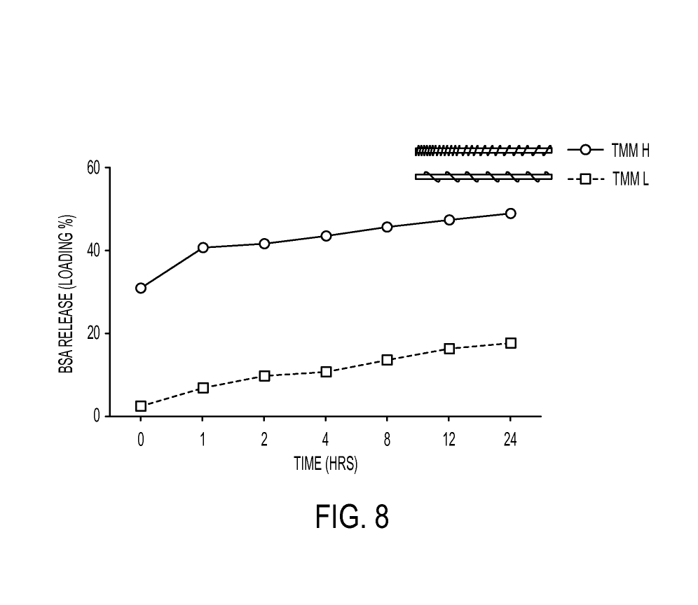

FIG. 8 is a graph showing BSA release over time for two regions of a coiled fiber. The graph shows a sustained released gradient in both a high (H) and low (L) coiled area during 24 hour testing period.

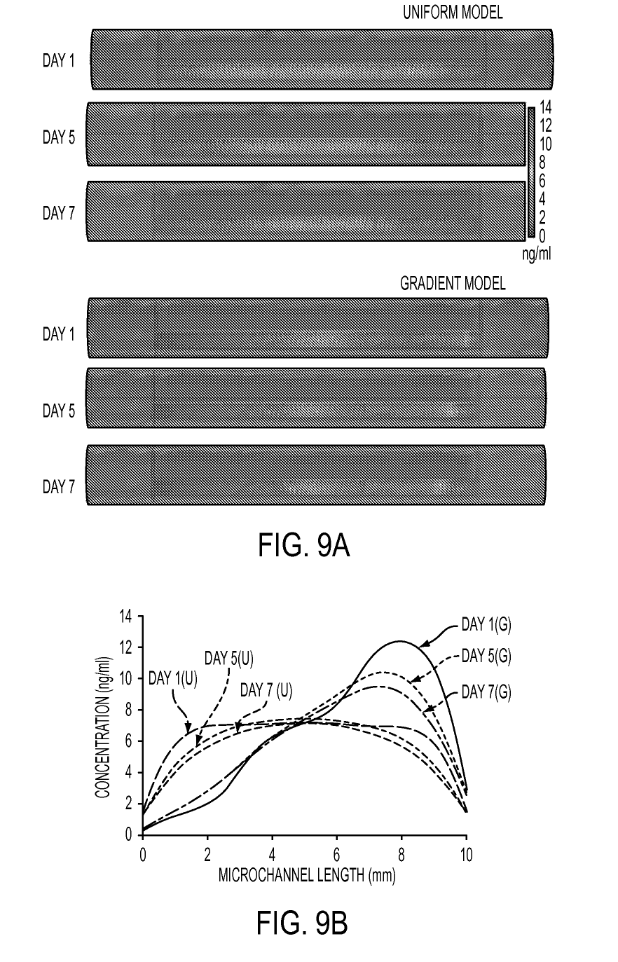

FIG. 9A is a graph comparing uniform and gradient distribution of coils after one, five, and seven days. The model-calculated NGF concentrations in the collagen-filled microchannel (volume ¼0.149 μl) at the end of 1, 5 and 7 days are presented for both uniform and gradient-coil configurations for comparison. For the uniform-coil configuration, the release of NGF is expected to be consistent through the entire channel. The channel concentration reached a level of 7.5 ng/ml at day 1, and remained at approximately 7 ng/ml at day 7. As a result of proximal and distal diffusion, the uniformity in NGF distribution gradually increases while NGF concentration decreases. It results in a high concentration zone in the center of the channel, which may discourage neurons from continuing growth toward the distal end (See FIG. 9A). In contrast, for gradient-coil configuration, the release of NGF and resulting concentration distribution in the channel is non-uniform by design. Our results show the NGF concentration varies from 0.02 to 12.42 ng/ml at day 1. At day 7, the NGF-concentration gradient is maintained with a minor decrease in the peak values to 9.53 ng/ml. The region of high NGF concentration corresponds to higher coil numbers. For the current set of design parameters, an average gradient of 0.02-10 ng/ml could be maintained over a week. Unlike the uniform-coil configuration, the gradient-coil configuration provides sustained chemotactic gradients which continue to entice and to guide the growth of neurons toward the channel distal end. These results support the notion that a favorable, sustainable molecular gradient of biologically-active growth factors can be established and maintained in the luminal collagen matrix by adjusting the number of coiled fibers on the walls of the agarose channels.

FIG. 9B is a graph of concentration versus microchannel length for uniform and gradient NGF after one, five, and seven days. FIGS. 9A and 9B show finite-element simulation of NGF diffusion from coiled fibers. FIG. 9A shows that the uniform distribution of the coils results in even diffusion of NGF across the microchannel over 1-7 days with some dilution at the openings. While the differential deployment of coiled fibers results in a 10-100 ng/mL NGF concentration gradient with a steep angle of 22°, which increases and expands over time to cover most of the volume of the microchannel. This difference is accentuated when the uniform and gradient concentrations are compared along the longitudinal axis.

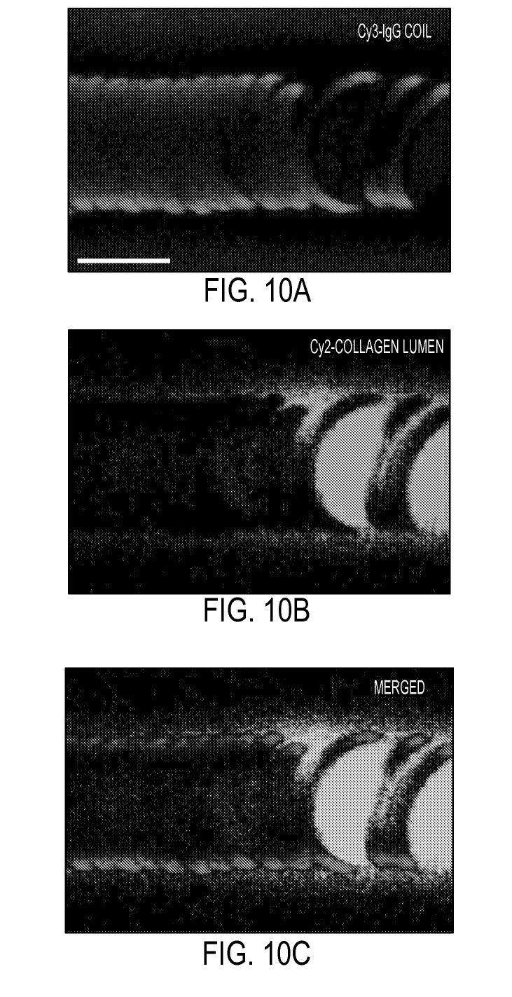

FIGS. 10A, 10B and 10C include three confocal images of a deployed polymeric Cy3-coil in agarose gel. Image (1) has been enhanced to show the Cy3-coil. The darker portion of the photo indicates the agarose, which is transparent. Image (2) has been enhanced to show Cy2-labeled collagen. Image (3) is a merger of image (1) and image (2). The bar shown in the bottom left corner of image (1) is a scale bar indicating a length of 100 μm.

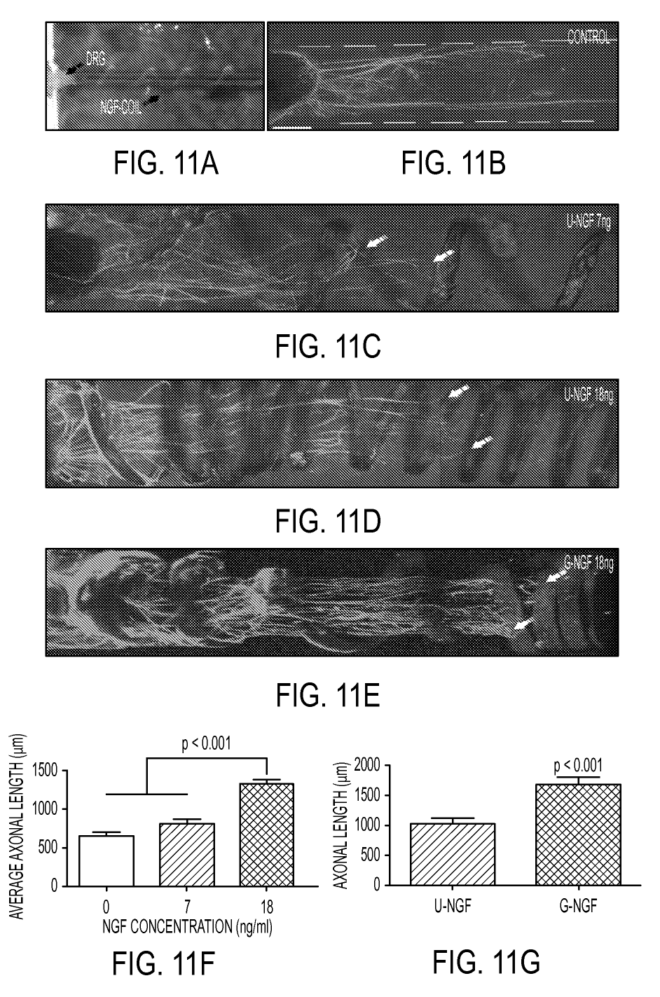

FIG. 11A is a DIC image of the TMM gel showing a microchannel with a DRG explant at the proximal end. FIGS. 11B-11E are confocal images of DRG axonal growth immunolabeled for β-tubulin visualization (lighter portions in FIGS. 11B-11E). FIG. 11B shows a control with no coils. The scale bars of FIG. 11B indicate a length of 100 μm. FIG. 11C shows coils of low-density NGF loaded fibers. FIG. 11D shows coils of high-density NGF loaded fibers. FIG. 11E shows coils of low-density NGF loaded fibers in a gradient configuration. Each of FIGS. 11C-11D shows an improved axonal growth compared to the growth shown in FIG. 11B.

FIG. 11F is a graph showing average axonal length at three NGF concentrations. An NGF concentration of around 7 ng/ml resulted in a slight improvement compared to the control, and an NGF concentration of around 18 showed a significant improvement in average axonal length compared to the control. FIG. 11G is compares average axonal length for uniform distribution of NGF and gradient distribution of NGF. The gradient distribution showed a significant increase in the average axonal length of neurons growing towards an increasing NGF concentration (n=3-5).

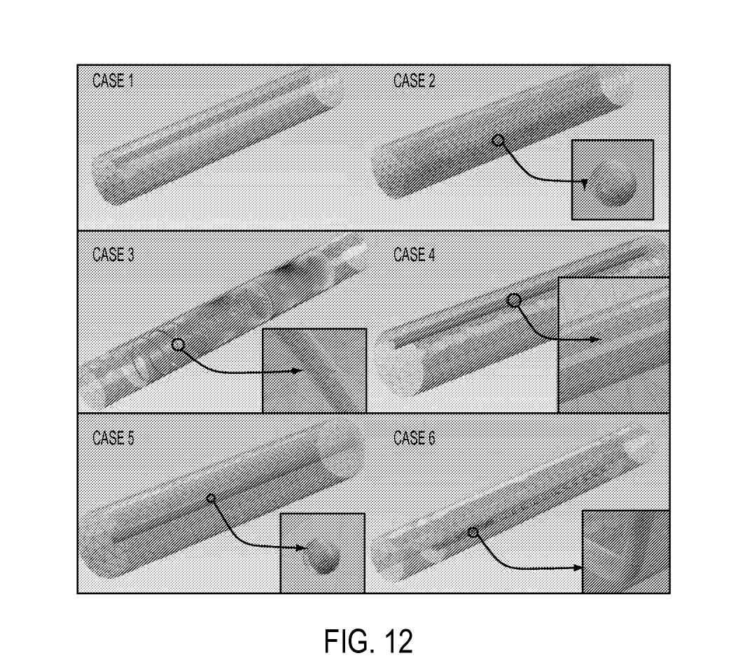

FIG. 12 shows meshed geometries depicting the different size of mesh elements. The critical geometry parts are zoomed in separately to make visualization easier due to the size difference.

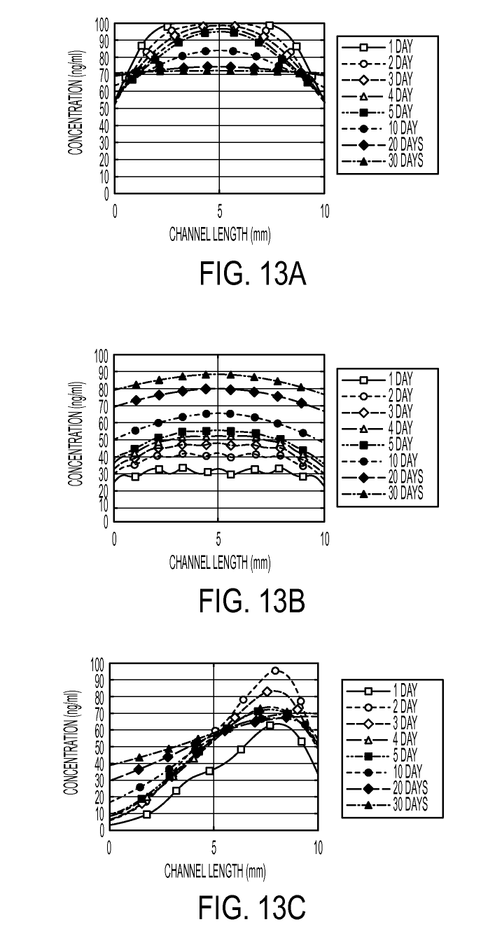

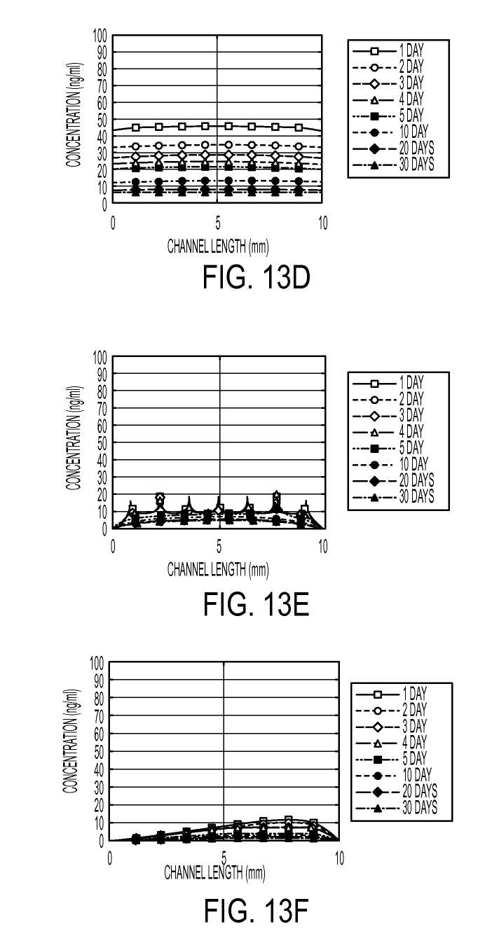

FIG. 13 includes graphs (1)-(6) that show axial concentration variation in an active channel/microchannel for six designs. For each of the graphs, concentration information was measured at 1, 2, 3, 4, 5, 10, 20, and 30 days. Graph (1) shows concentration vs channel length for homogenously mixed GF. Graph (2) shows concentration vs channel length for GF packaged into homogenously distributed PLGA microspheres. Graph (3) shows concentration vs channel length for GF packaged into differently coiled fiber. Graph (4) shows concentration vs channel length for homogenously mixed GF in microchannel. Graph (5) shows concentration vs channel length for GF packaged into homogenously distributed PLGA microspheres in microchannel. Graph (6) shows concentration vs channel length for GF packaged into differently coiled fiber inside microchannel.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A method of promoting spinal cord growth or regrowth in a subject comprising:
implanting a conduit comprising a tubular body into a site of spinal cord deficit; and
wherein a central axis of the conduit is positioned such that a lumen of the conduit is generally parallel to an axis of desired nerve growth or regrowth.

2. The method of claim 1, further comprising anchoring the conduit between adjacent vertebral segments.

3. The method of claim 1, further comprising securing the conduit in the site by suturing adjacent facial sheets or muscle fibers.

4. The method of claim 1, wherein said conduit is composed of poly-lactide acid, poly-urethane, polydioxanone, silicone, cellulose, collagen, PLGA, polycaprolactone or denatured natural extracellular matrix.

5. The method of claim 1, further comprising a luminal filler (LF) disposed in the lumen of said conduit.

6. The method of claim 5, wherein the LF is comprises one or more of agar, collagen, laminin, fibronectin, or glycoprotein.

7. The method of claim 5, wherein the LF comprises a microchannel formed through lumen of the conduit.

8. The method of claim 5, wherein the LF comprises a nerve-growth factor.

9. The method of claim 5, further comprising:
wherein the LF comprises microparticles or eluting fibers; and
wherein the microparticles and eluting fibers comprise a nerve-growth factor.

10. The method of claim 9, wherein said nerve-growth factor elutes over time.

11. The method of claim 1, wherein an outer surface of the conduit is coated with a nerve-growth factor.

12. The method of claim 11, wherein the nerve-growth factor includes one or more of neurotrophic (NGF, BDNG, NT-3), glial-derived (GDNF), and pleotrophic (PTN, VEGF).

13. The method of claim 5, wherein the LF comprises a nerve-growth factor that is distributed along a length of the conduit with a gradient of dense nerve-growth factor to less dense nerve-growth factor.

14. The method of claim 5, wherein the LF comprises a nerve-growth factor that is distributed along a length of the conduit with a bidirectional gradient of one or more nerve-growth factor(s).

15. The method of claim 1, wherein said conduit is from about 0.5 mm to about 5 mm in length.

16. The method of claim 1, wherein said conduit is from about 1.5 mm to about 14.0 mm in outside diameter.

17. The method of claim 1, wherein the lumen of said conduit is from about 1.5 mm to about 13.0 mm in diameter.

18. The method of claim 1, wherein the conduit wall is about 0.2 mm to about 0.6 mm in thickness.

19. The method of claim 1, wherein the conduit or implantation site is treated with collagen.

20. The method of claim 1, wherein the axis of desired nerve growth is parallel to an axon of the site of spinal cord deficit.

21. A method of treating a tissue deficit in a subject comprising surgically implanting a conduit, medical implant or polymeric regenerative guide fibers into a site of nerve deficit of a subject.

22. The method of claim 21, wherein said nerve deficit is in the brain, spinal cord or peripheral nerve.

23. The method of claim 21, wherein the tissue is meninges or dura.

24. The method of claim 21, wherein medical implant is an electrode, deep brain stimulator, a pump or an antenna

25. A device for releasing a drug comprising:
a conduit comprising a lumen;
a luminal filler disposed within the lumen, the luminal filler comprising one or more channels disposed through the conduit;
a fiber disposed within the one or more channels.

26. The device of claim 25, wherein the fiber is coiled along a length of the one or more channels.

26. The device of claim 26, wherein the fiber comprises a first portion having a first set of coils spaced a first distance apart from one another, and a second portion having a second set of coils spaced a second distance apart from one another.

28. The device of claim 26, wherein a pitch of the coiled fiber changes along the length of the microchannel.

29. The device of claim 25, wherein the fiber comprises neurotrophic factors.

30. The device of claim 25, wherein the fiber comprises a nerve-growth factor.

31. The device of claim 25, wherein the lumen comprises collagen.
